(19) United States
(12) Patent Application Publication
(10) Pub. No.: US 2012/0265080 A1
(43) Pub. Date: Oct. 18, 2012

(54) NON-CONTACT SENSING OF PHYSIOLOGICAL SIGNALS

(76) Inventors: Xiong Yu, Beachwood, OH (US); James Berilla, Cleveland, OH (US); Ye Sun, Cleveland, OH (US)

(21) Appl. No.: 13/447,923

(22) Filed: Apr. 16, 2012

Related U.S. Application Data
(60) Provisional application No. 61/476,042, filed on Apr. 15, 2011.

(51) Int. Cl. A61B 5/0205; A61B 5/0402; A61B 5/053

(52) U.S. Cl. 600/484; 600/547; 600/509

(57) ABSTRACT

A non-contact monitoring system can include an electrode configured to detect electrical signals from a surface of a subject's body without directly contacting the surface of the subject's body (e.g., via capacitive coupling). The electrode can be positioned at a spaced apart distance from the subject's body (e.g., ranging up to about 30 cm). The signals from the electrodes can be processed in the analog and digital domain to determine one or more physiological conditions of a subject, such as drowsiness.

### CROSS-REFERENCE TO RELATED APPLICATION

This application claims the benefit of U.S. Provisional Patent Application No. 61/476,042, filed Apr. 15, 2011 and entitled APPARATUS AND METHOD FOR NON-CONTACT SENSING OF PHYSIOLOGICAL SIGNALS, which is incorporate herein in its entirety.

### GOVERNMENT FUNDING

This invention was made with government support under Grant No. RES503350 awarded by The Ohio Board of Regents. The United States government may have certain rights to the invention.

### TECHNICAL FIELD

This disclosure relates to sensing of electrical signals and, more particularly to non-contact capacitive sensing of electrophysiological signals.

### BACKGROUND

Traffic accidents are projected to be the third leading cause of death and disability in 2020. Driver fatigue is a leading cause of traffic accidents. For example, the Federal Motor Carrier Safety Administration (FMCSA) issued regulations on the Hours of Service (HOS) in order to prevent accidents caused by driver fatigue. These rules regulate the minimum time drivers must spend on resting between driving shifts. However, since different drivers have different physical and mental conditions, safety risks still remain.

Generally, driver fatigue impairs cognitive skills and reduces the vigilance and attention of drivers to continue driving safely. Assessment of driver fatigue can be divided into two categories, subjective methods and objective methods. The subjective assessment is based on the state of drivers described by themselves. For example, special-purpose questionnaires can be used before, during or after driving, to obtain information about fatigue experienced by a given driver. Due to the differences in individuals, privacy and the effects of environments, the accuracy of subjective assessment cannot be guaranteed.

### SUMMARY

This disclosure relates to an apparatus and method for non-contact sensing of physiological signal.

As one example, a non-contact physiological monitoring system can include a non-contact electrode configured to provide an input sensor signal based on electrical activity at a subject's body. The electrical activity can be capacitively coupled to induce current on the non-contact electrode without contacting the surface of the subject's body. An instrument amplifier can amplify the input sensor signal to provide an amplified input signal. A DC bias circuit can be configured as a high-pass filter to substantially remove DC offset in the input amplified input signal and provide an offset-corrected signal. A high order analog low pass filter in series with the DC bias circuit can be configured to pass frequency content below a predetermined cut-off frequency and to apply a gain factor to the offset-corrected signal and provide a corresponding analog output signal at an output representing the electrical activity at the subject's body, the gain factor being greater than about 500.

As another example, a system can include a plurality of non-contact electrodes, each of the electrodes being configured to capacitively couple with an adjacent region of a subject's body that is spaced apart from the respective electrode and to provide a respective output signal corresponding to electrical activity sensed at the adjacent region via the capacitive coupling. An analog circuit can be configured to amplify and filter each respective output signal and provide an analog output signal. A processing device can be configured to process each analog output signal. The processing device can include a digital filter programmed to filter a digital representation of each analog output signal and provide processed signals corresponding to each of the analog output signals. The processing device can also include a calculator to determine a plurality of physiological conditions for the subject based on the processed signals. An output generator can be configured to provide an output based on the plurality of physiological conditions for the subject.

As yet another example, a non-contact method for monitoring physiological conditions can include inducing electrical current on at least one electrode, which that is spaced apart from an adjacent region of a subject's body, via capacitive coupling between the respective electrode and the adjacent region of the subjects body. At least one electrical signal is received at an input corresponding to the induced electrical current. The electrical signal can be amplified and filtered in the analog domain and a corresponding analog output signal can be provided in which DC bias has been substantially removed as to mitigate saturation of the corresponding analog output signal for a distance between the at least one electrode and the adjacent region of the subject's body that is up to about 30 cm. A digital representation of the corresponding analog output signal can be digitally filtered to remove noise and provide a processed signal corresponding to the corresponding analog output signal. At least one physiological condition for the subject can be determined based on the processed signal and an output can be generated based on the at least one physiological condition.

### BRIEF DESCRIPTION OF THE DRAWINGS

FIG. 1 illustrates an example of a non-contact sensor system.

FIG. 2 illustrates an example of a circuit that can be implemented in the sensor system of FIG. 1.

FIG. 3 depicts a signal to noise ratio for different distances between a non-contact electrode and a subject.

FIGS. 4A, 4B and 4C illustrate an example of ECG signals detected off body through clothing at different distances.

FIGS. 5A and 5B illustrate example electrophysiological signals detected from different parts of human body.

FIG. 6 depicts an example of a drowsiness detection system.

FIGS. 7A and 7B illustrates an example of ECG signals that can be determined from non-contact sensing.

FIGS. 8A and 8B illustrate an example of comparing input and output signal of processing.

FIG. 9 illustrates an example of a breathing pulse signal derived from an ECG signal.

FIG. 10 illustrates an example of a muscle activity signal detected from non-contact sensing.

### DETAILED DESCRIPTION

### Overview

This disclosure relates to sensing of an electrical physiological signal via non-contact capacitive coupling between an electrode and a subject.

A non-contact monitoring system can include an electrode configured to detect electrical signals from a surface of a subject's body without contacting the surface of the subject's body (e.g., via capacitive coupling). The electrode can be positioned at a distance of greater than 5 cm (e.g., ranging up to about 30 cm) from the subject's body. A sensor circuit is coupled to the electrode, the sensor circuit being configured to amplify and filter the detected electrical signal and provide a corresponding analog output signal that includes a physiological signal for the patient as well as some noise. A digital processing module can further filter the amplified signal to remove noise and provide a processed signal representing the desired physiological signal. An adequate signal to noise ratio (SNR) can be provided even at distances of up to 30 cm.

One or more non-contact sensors can be utilized to detect the physiological signals for different parts of the body and may be positioned at different distances from the body surface being monitored. In some examples, the system can also include one or more calculator configured to compute one or more physiological condition from the processed signals, such as heart rate, heart rate variation, breathing rate or muscle activity of a particular structure (e.g., corresponding to eye movement).

As disclosed in the examples herein, the non-contact sensor provides a platform technology that affords significant flexibility since it can be utilized in a variety of applications. Example applications that may utilize such a non-contact sensor platform can include transportation safety, breathing problems, cardiac patients, neonatal and infant monitoring and burn victims. As one example, the remotely detected physiological signal(s) can be analyzed to determine an indication of driver fatigue for a person driving a vehicle (e.g., car, truck, train, plane, boat, etc.).

### Description of Example Embodiments

This disclosure describes an apparatus and method for non-contact detection of physiological signals of a subject. In some examples, this disclosure describes the apparatus and methods in the context of transportation safety application, such as implemented in a vehicle to provide an objective indication of driver fatigue. However, the apparatus and methods disclosed herein are not limited to this context as it can be employed for a variety of other applications, such as disclosed herein.

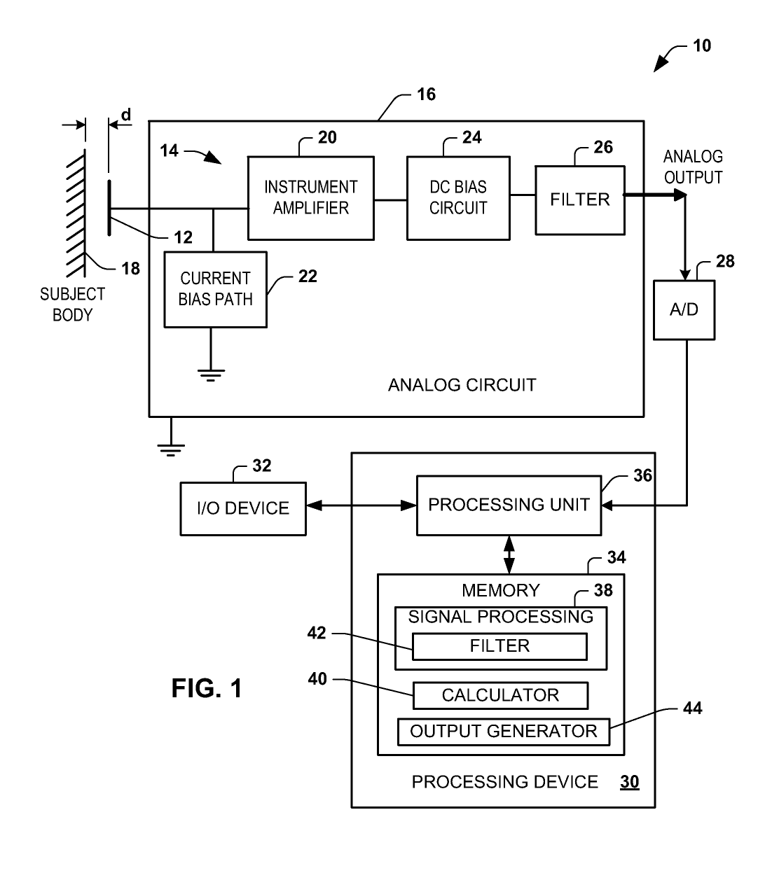

FIG. 1 depicts an example of a sensor system 10 that provides for non-contact sensing of electrophysiological signals from a subject's body 18. The sensor system 10 can be implemented in a vehicle, for example. The vehicle can be an automobile or truck, an aircraft, a watercraft or train. Alternatively, the sensor system can be utilized for a variety of other monitoring applications, such as a hospital (e.g., infants, burn victims), home monitoring of a patient or other applications in which non-contact monitoring of a subject's electrophysiological conditions may be desired.

The sensor system 10 includes an electrode 12 of an electrically conductive material that provides an input electrical signal to an analog circuit 14. The electrode 12 can be implemented as an electrically conductive plate, such as a metal plate, an electrically conductive polymer or a combination of different conductive materials. Other example materials for the electrode 12 can include copper, silver, iron, aluminum and the like.

As an example, the electrode 12 can be formed of a plate having an electrically conductive surface area such as ranging from about two centimeters squared to about nine centimeters squared. In some examples, the electrode structure can be substantially rigid. In other examples, a flexible electrode structure (e.g., a copper foil or an electrically conductive cloth or fabric) can be utilized. For example, where the electrode is to be positioned in close proximity to the subject's body 18, flexible electrically conductive polymers can be utilized since it can reduce discomfort due to contact and attachment to clothing or furniture (e.g., chairs, beds or the like), on which the body surface may be positioned during sensing.

The sensor electrode 12 can be positioned in a spaced apart relationship from the surface of the subject's body 18 (e.g., the body surface can be spaced up to about 30 centimeters from the sensor plate). The distance can be fixed or it can vary such as in response to movement of the subject relative to the electrode. Electrical signals at or near the surface of the body 18 capacitively couple to the electrode 12 to provide the corresponding input signal to the analog circuit 14. While the example system 10 of FIG. 1 depicts a single electrode 12, there can be any number of one or more such sensor plates, each of which can have corresponding circuitry for providing a corresponding analog output signal. Additionally, as used herein, non-contact means that the electrode does not directly contact the subject's body. However, in some examples, clothing or other materials may be interposed between the subject's body and the electrode. The materials along with any air operate as dielectrics in the capacitive coupling.

The analog circuit 14 can be positioned within a sensor housing 16. The housing 16 can be formed of a material to shield the analog circuit 14 therein from electrode magnetic or other interference. For example, the shielded housing 16 can be formed of an electrically conductive material that is electrically coupled to a signal ground to mitigate interference with the analog signals propagating through the analog circuit 14. The electrode 12 can be fixedly mounted to the housing 16, such as attached to a corresponding side surface thereof. The mounting to the exterior surface provides a convenient implementation for examples where room exists for mounting the housing and the corresponding analog circuit 14 together. In other examples, the electrode 12 can be mounted at any location that is spaced apart from and electrically connected with the analog circuit 14, such as through a shielded cable (e.g., a coaxial cable).

In examples where the system 10 includes multiple sensor electrodes 12 distributed at different sensing locations, the analog circuit 14 can be contained in a single housing 16 electrically coupled to each electrode. Alternatively, separate housings can be utilized, such as depending on the relative location of the electrode plates and other design considerations.

By way of example, neural activity due to muscle activity (e.g., of the heart or other muscles can create electrical potentials at or near the surface of the subject's body. The non-contact electrode 12 can detect the electrical activity from the subject's body caused by flowing charges via capacitive coupling. For instance, the electrode 12 and the subject's body 18 operate as a coupling capacitor. In many examples, the dielectric spacer between the electrode 12 and the subject's body 18 is air, clothing or other known materials. Due to the capacitive coupling, the charges on the subject's body 18 can induce electrical current in the electrode in proportion to the electrical activity that is being detected. In this way, the sensor electrode 12 can operate as a remote non-contact device to sense the electrical signals on the patient's body to determine one or more physiological condition for the subject based on processing performed by the analog circuit 14 and subsequent digital processing as disclosed herein.

As a further example, the electrode 12 can be positioned near a patient's chest such as a front or rear portion of the chest (torso) to detect an electrocardiogram (ECG) signal or other signal corresponding to cardiac electrical activity. The electrode 12 can also be positioned to detect other electrical activity corresponding to muscle activity, such as in the form of an electromyogram (EMG). The other muscle activity can be associated with eye blinking or activation of other muscle fibers in proximity to the electrode. As yet in another example, one or more sensor plates can be positioned adjacent a patient's head in a non-contact arrangement to detect signals corresponding to an electroencephalograph (EEG) corresponding to brain electrical activity. The circuit 14 and subsequent digital processing by processing device 30 can provide an indication of the sensed electrophysiological activity as well as derived indications of other physiological conditions derived from processing of the sensed electrical signals (e.g., heart rate, breathing rate, body movement and the like).

Returning to FIG. 1, the analog circuit includes an instrument amplifier 20. The sensed voltage signal at the electrode 12 is electrically coupled to an input of the instrument amplifier 20. A current bias path 22 can also be provided at the input to the amplifier 20 to facilitate converting the capacitive coupled input signal to a corresponding voltage at the input of the amplifier.

As an example, the input impedance at the amplifier 20 can be about 10¹⁸Ω. Depending on the distance between the electrode 12 and subject's body 18, noise as a common mode signal may have greater amplitude than the electrophysiological signal that is received as a differential mode signal at the input. Accordingly, the amplifier 20 can be configured with high common mode rejection ratio (CMRR). In one example implementation, the amplifier 20 that performs the first amplification of the signal can be completed by an instrumentation amplifier (e.g., INA116 amplifier that is commercially available from Texas Instruments Incorporated). Such amplifier, for example, can have CMRR of about 90 dB at 0-1 kHz when the gain is about 10V/V.

The output of the amplifier 20 is provided to a DC bias circuit 24. The DC bias circuit 24 can be implemented as a high pass filter having a low cutoff frequency (e.g., about 0.5 Hz) to help remove DC offset. By removing DC offset in this manner, saturation of the amplified signal (including further amplification and filtering) by the analog circuit 14 can be mitigated. The DC bias circuit 24 in turn provides a corresponding offset-corrected signal to a filter 26. The filter 26, for example, can be implemented as a high order low pass filter with a high gain coefficient (e.g., a gain greater than or equal to about 500). As used herein, a high-order low pass filter corresponds to an order of filter that is three or greater. For example, the filter 26 can be implemented as a fourth order low pass filter and provide a gain of about 1000. The cut-off frequency of the filter 26 can be set to about 45 Hz such that the resulting filtered output signal corresponds to desired electrophysiological parameters. The filter 26 thus provides a corresponding filtered and amplified analog output signal to an analog-to-digital converter (A/D) 28.

As a further example, the analog output signal provided by the filter 26 can have a peak-to-peak amplitude that is greater than or equal to about 0.5 V (e.g., about one volt). For example, the analog circuit 14 can provide an aggregate gain that exceeds 1000 (e.g., ranging between about 4000 and about 6000) such that the peak-to-peak amplitude of the voltage signal can be greater than or equal to about 0.9 V for distances of up to about 25 cm between the electrode 12 and the subject's body 18. Despite the quantity of noise that is received via the electrode 12 and the high gain implemented by the analog circuit 14, the analog output signal still can provide sufficient information for detecting physiological parameters of the subject and avoid saturation.

Since the analog output signal still contains noise, the corresponding digitized signal can be provided to the processing device 30 to perform additional filtering and de-noise such signals. The processing device 30 can be implemented as part of a computer or an otherwise special processing device (e.g., a digital signal processor or an ASIC). In the example of FIG. 1, the processing device 30 includes a memory and a processing unit 36. The memory can store data and executable instructions for performing functions and methods disclosed herein. The processing unit 36 can access the memory 34 and execute instructions that are stored in the memory. The instructions can include a signal processing method 38 programmed for processing the digitized signal. The signal processing can include a digital filter function 42 that can be programmed as a bandpass filter. For instance, the filter function 42 can be implemented as a high order digital filter implemented in the software that is tuned to pass the desired frequency band corresponding to the physiological condition being monitored. For example, the bandpass filter can be programmed with a pass band ranging between about 0.5 and about 40, Hz such as for detecting cardiac electrical activity corresponding to an ECG.

The memory 34 can also store instructions corresponding to one or more calculators 40. The calculator 40, for example, can be programmed to compute an indication (e.g., a value) representing one or more physiological conditions for the subject based on the filtered signal. The filter 42 thus can be programmed with different filter parameters functions according to the signal content and the type of physiological condition being detected. Example conditions that can be computed by the calculator 40 can include heart rate, heart rate variability, brain activity, breathing rate and eye blinking rate to name a few. As disclosed herein, heart rate refers to the number of heartbeats per unit of time.

Additionally or alternatively, the calculator 40 can be programmed to derive electrophysiological signals corresponding to the types typically monitored by contact sensors, such as an ECG signal, an EEG signal or the like. For instance, the calculator 40 can operate as a waveform generator programmed to convert the processed signals, which were sensed via the non-contact electrode, to a form consistent with that utilized by healthcare professionals for diagnostic purposes. As an example, by identifying known attributes of an ECG waveform (e.g., a P wave, a QRS complex, a T wave, and a U wave and associated intervals), the calculator 40 can remove extraneous signal content (e.g., via wavelet transform) and in turn generate a corresponding ECG waveform based on the processed signal detected from electrical activity from a subject's chest.

The memory 34 can also include an output generator 44 that is programmed to provide a corresponding output. The output can vary depending upon which one or more physiological condition is being monitored and the function of the calculator 40. The output generator 44 further can provide an indication of the sensed condition or it can provide a representation of the waveform, which can be interpreted by a user such as via an input/output device 32. The input/output device 32 can include a display, an audible indicator (e.g., a speaker) or other device that can provide information to a user. In other examples, the input/output device 32 can wirelessly transmit data to one or more remote destinations (e.g., via WiFi, cellular data or the like). The transmitted data can be received and interpreted by another user, such as on a personal computer, laptop, smart phone or tablet computer, for example.

As one example, the calculator 40 can be programmed to determine an indication of drowsiness or fatigue based upon the capacitively coupled signals received and processed by the analog circuit 14 and the processing device 30. For example, the calculator 40 can be programmed to compute an indication of drowsiness based on two or more of a detected heart rate, heart rate variability, breathing rate, eye blinking rate and brain wave activity. The output generator 44 can in turn provide a corresponding indication of the drowsiness based on the calculated physiological conditions determined from the processed signals. A heart rate signal can provide an overall indictor that reflects the physical and mental condition of a person under different task requirements. For instance, heart rate signal can indicate the combined effect of tasks, feelings, etc., on vehicle operators. It has been shown that heart rate of drivers tends to decrease during long-time night driving, and that fatigue has significant effects on the change of heart rate.

In the example, where the detection and calculations are performed in a vehicle in real time, the input/output device 32 can provide corresponding countermeasures in response to the determined indication of drowsiness. For example, the counter measures can include one or more of an audible warning (e.g., a beep, or alarm, adjusting vehicle radio controls), a visual warning (e.g., a flashing light) and/or a tactile counter measure (e.g., a vibration, control vehicle fans for airflow) or the like. The input/output device 32 can be existing equipment on the vehicle or it can be specially added equipment as part of a driver fatigue warning system that includes the sensor system 10.

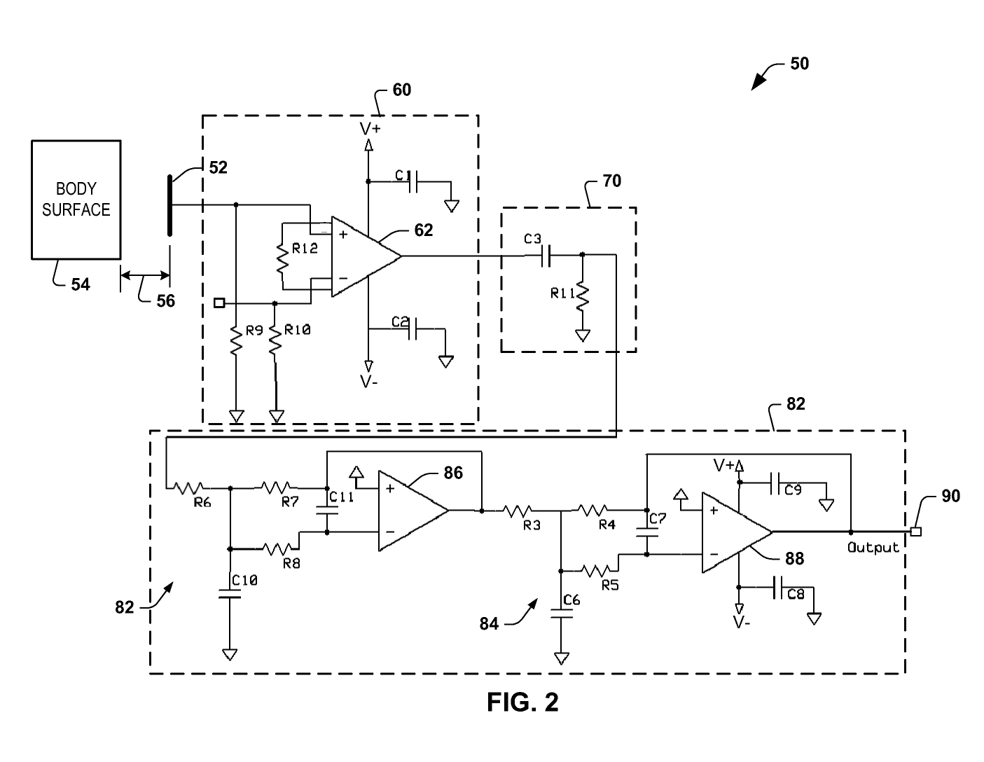

FIG. 2 depicts an example of analog circuitry 50 that can be utilized to acquire electrophysiological signal from a body surface 54. For example the analog circuit 50 can correspond to the analog circuit 14 disclosed with respect to FIG. 1. An electrically conductive electrode 52 is positioned at a distance, indicated at 56, from the body surface 54. The electrode 52 provides a non-contact sensor that is spaced apart from the body surface 54 by the distance 56. The analog circuit 50 is configured to provide a discernible signal corresponding to the sensed physiological condition (e.g., having a sufficient SNR) at the body surface 54 for a distance 56, such as up to about 30 cm.

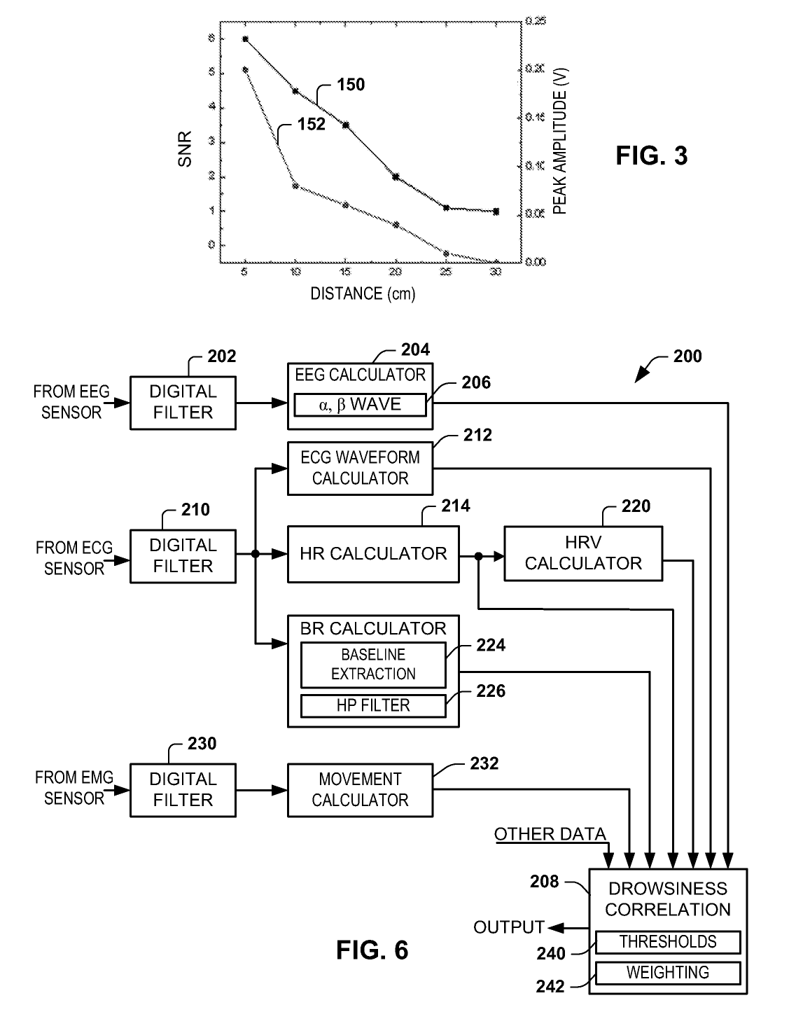

FIG. 3 depicts an example of a graph illustrating SNR 150 and peak amplitude 152 of signals detected by a non-contact sensor, as disclosed herein, for a source of electrical activity at different distances. For example, a source device was placed in front of the electrode with the frequency 10 Hz and peak amplitude 20 μV. As the device was moved forwards and backwards, the amplitude 152 of output changes with the distance between the source and the electrode. Every 5 cm, the peak amplitude and the SNR was recorded. For distance range between 25 cm and 30 cm, the signal is still detectable but inundated in noise. As shown, for the example signals detected, the SNR decreases apparently with the distance from body. At distances less than 20 cm, the sensor can clearly detect the signal.

Returning to FIG. 2, the physiological signal on the body surface is capacitively coupled to the electrode 52 to provide a corresponding voltage at an input of an amplifier 60. The amplifier 60 can include a current bias path, including a resistor R9, coupled between the non-inverting input and ground. The inverting input can remain unused as demonstrated in the example of FIG. 2 in that it is not coupled to an electrode. In other examples, another electrode may be coupled to the inverting input. A resistor R10 is connected between the inverting input and ground. A gain resistor R12 is also connected to inputs of the amplifier 60 to set the gain of the amplifier. As one example R12 can be set to 10K ohms such that the gain (e.g., gain=1+50 k ohm/R12) can be about 6. In other examples, the gain range from about 4 to about 7 based on selecting the resistor R12. The op amp 62 can be coupled between voltage rails indicated at V+ and V− which sources can be coupled to ground via filtering capacitors C1 and C2.

The output of the op amp 62 can be provided to a DC bias circuit 70. For example, the bias circuit 70 can be configured as a high pass filter and include a combination of the capacitor C3 and a resistor R11 coupled between C3 and ground. The output of the DC bias circuit 70 can be provided to a low pass filter 80. The low pass filter 80 can correspond to a high order low pass filter (e.g., a fourth order low pass filter). Thus the series combination of the DC bias circuit 70 and the low pass filter 80 collectively form a bandpass filter. The series combination further can be configured with a pass band corresponding to the requirements of the signal being detected and provide a gain of about 1000 within the pass band. For example, the corresponding band pass filter defined by the DC bias circuit 70 and the low pass filter 80 can provide a gain of about 1000 with a −3 dB cutoff frequency in a pass band of about 2 Hz to about 30 Hz. Thus, the low pass filter 80 itself can be configured to have a −3 dB cutoff frequency of about 30 Hz.

In the example of FIG. 2, the high order low pass filter 80 can be implemented as a series combination of a two low pass filter stages 82 and 84. Each of the filter stages 82 and 84, for example, can be implemented as a corresponding second order filter such that the aggregate low pass filter 80 that defines a fourth order low pass filter. A non-inverting input of the first stage low pass filter 82, can be coupled to receive the offset-corrected output from the DC bias circuit 70. The offset corrected signal is provided to an arrangement of resistors and capacitors. For example, an input resistor R6 is coupled to a capacitor C10 coupled to ground and across which a parallel combination of R7 and C11 are in parallel with resistor R8 to drive an inverting input of an op amp 89. A feedback path connects the output of the op amp 86 to the node between R7 and C11.

The output of the op amp 86 drives the second stage filter 84, which includes a similar combination of components including R3, R4, R5, C6 and C7 which electrically connect the output of op amp 86 to the inverting input of op amp 88. Filtering capacitors C8 and C9 can be coupled to biased voltages V+ and V− of the op amp 88. It is to be understood that the particular order of the low pass filter stages 82 and 84 can be switched such that the filter stage 84 is connected to the DC bias circuit 70 and the filter stage 82 is coupled between the second stage and the output at 90. The following table provides list of example components that can be implemented in the analog circuit 50. It is to be understood and appreciated that other component values could be implemented depending upon application requirements, for example.

TABLE 1

| COMPONENT | VALUE | |
|---|---|---|
| C1 | 0.1 | μF |
| C2 | 0.1 | μF |
| C3 | 470 | μF |
| C6 | 2 | μF |
| C7 | 0.15 | μF |
| C8 | 0.1 | μF |
| C9 | 0.1 | μF |
| C10 | 2 | μF |
| C11 | 0.0068 | μF |
| R3 | 1.69 | KΩ |
| R4 | 84.5 | KΩ |
| R5 | 11 | KΩ |
| R6 | 4.22 | KΩ |
| R7 | 84.5 | KΩ |
| R8 | 24.3 | KΩ |
| R9 | 100 | MΩ |
| R10 | 100 | MΩ |
| R11 | 200 | KΩ |

As disclosed herein, the non-contact sensing approach can detect various electrophysiological signals of a subject, such as including electrocardiography signals, electroencephalograph signals. It can also measure behaviors and body movement such as can include head movement, eye movement or the like, based on non-contact sensing of electromyography signals.

Physiological signals, such as electrophysiological signals can provide good indicators of fatigue. It is generally believed that driver fatigue is the nature of central nervous system. When stress response of organs occurs during fatigue, cardiovascular nervous system will adjust accordingly. Examples of the frequency and magnitude of common bioelectrical signals that can be detected by a non-contact capacitive sensing system are shown in Table 2. These examples can include electrocardiography (ECG), electroencephalography (EEG), electromyography (EMG), electro (EOG) and electroglottography (EGG).

TABLE 2
The magnitude and frequency of typical bioelectrical signals

| Bioelectrical Signal | Magnitude | Frequency |
|---|---|---|
| ECG | 50 μV-50 mV | 0.05 Hz-100 Hz |
| EEG | 2 μV-10 μV | 10 Hz-2 kHz |
| EMG | 20 μV-10 mV | 10 Hz-10 kHz |
| EOG | 10 μV-4 mV | 0.1 Hz-100 Hz |
| EGG | 10 μV-80 mV | 0 Hz-1 Hz |

Electrical charges on skin are caused by the depolarization of heart muscles during each cardiac cycle (e.g., each heart beat). Depolarization is a phenomenon that reduces the polarity towards zero in each heart muscle cells and causes it to contract. During each heartbeat, nerve excitability is triggered at sinoatrial node, and then spreads through atrium, intrinsic conduction pathways and ventricles sequentially. As a result of this process, it causes the change of action potential in cells manifested as the form of tiny rises and falls of potential on body surface. The electrical activity of heartbeat cycle is adjusted rhythmically by the central and peripheral nervous system. During fatigue, there will be some change in spontaneous rhythmic activity, breathing, cardiovascular reflex activity, etc. The comprehensive regulation of these changes impacts heart rate (HR). Therefore, monitoring ECG signal and other electrophysiological conditions, as disclosed herein, can be utilized to characterize the fatigue state of drivers.

As an example, methods for characterizing fatigue in a driver based on ECG signal can include Heart Rate (HR) analysis, Heart Rate Variability (HRV) analysis and amplitude of T wave, which is an interval of an ECG period. As disclosed herein, HR refers to the number of heartbeats per unit of time. HR signal can provide an overall indictor that reflects the physical and mental condition of a person under different task requirements. For instance, HR signal can indicate the combined effect of tasks, feelings, etc, on operators. It has been shown that HR of drivers tends to decrease during long-time night driving, and that fatigue has significant effects on the change of HR.

Heart rate variability (HRV) provides a measure of variations in the heart rates. HRV can also be related to fatigue. It has been shown that 0.1 Hz HRV was a sensitive indicator of driver fatigue; however, the spectrum of HRV can vary significantly during the conditions of fatigue driving. HRV can also be utilized to indicate the workload of human cognitive condition.

The relationship between HR and HRV on a given person can vary depending on mental and physical loads on such person. The two kinds of loads (mental and physical) can further influence each other and in turn affect HR and HRV. In some cases, when mental workload increases, HRV decreases while the HR tends to remain substantially the same. In other cases, it has been found that with load rising up, HRV can decrease and HR can also increase significantly. In other studies, as physical load became heavier, HRV would lessen and HR would increase, as expected.

The apparatus and methods disclosed herein can detect ECG in a non-contact manner and derive an indication driver fatigue. In order to achieve this reliably and effectively, reliable ECG signals are acquired by sensors and efficient signal processing is implemented. As disclosed herein, the apparatus and methods can detect the ECG signal of a person (e.g., a driver of a vehicle) at a distance up to 30 cm through clothes.

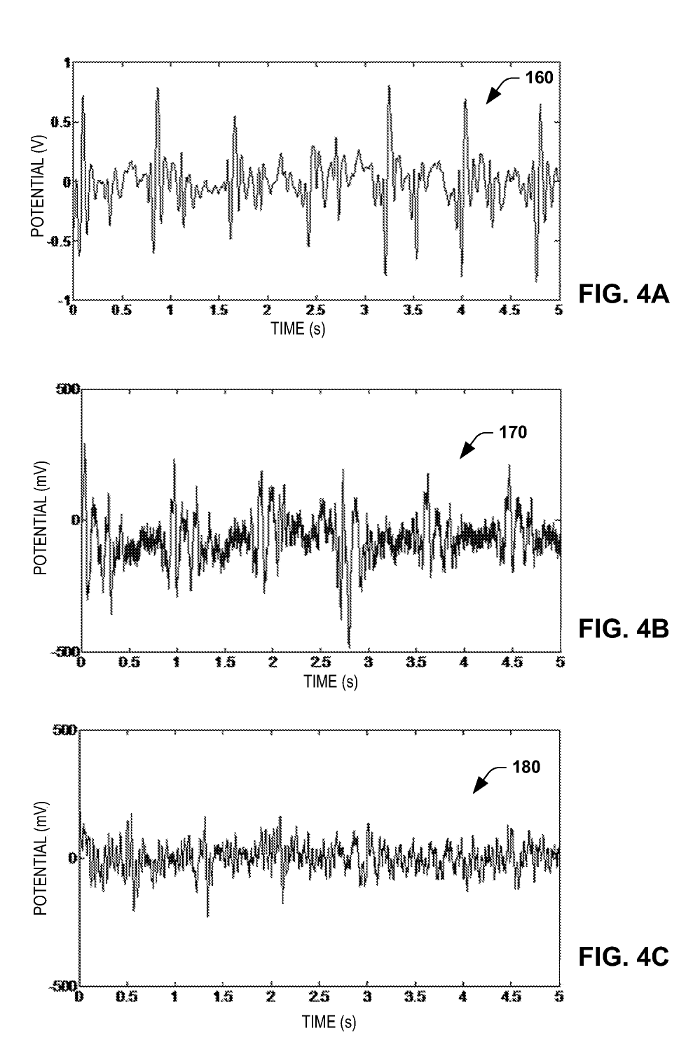

By way of example, FIGS. 4A, 4B and 4C demonstrate signals obtained via non-contact sensing in experiments conducted in a noisy unshielded room. During the experiment, the subject was seated in a chair, and a sensor electrode was positioned off body in front of left chest through clothing at distances of 10 cm, 20 cm, 30 cm, respectively to obtain different data shown at 160, 170 and 180 in FIGS. 4A, 4B and 4C, respectively. The conditions of the three signals were the same except the distance between chest and sensor. In FIGS. 4A, 4B and 4C, the amplitude of ECG signal becomes smaller and the noise becomes greater with the distance increasing. This is consistent with the SNR data demonstrated in the example of FIG. 3. When the distance is less than 20 cm, the sensor can detect the ECG signal clearly. If the distance exceeds 25 cm and less than 30 cm, the signal can still be detected, but the resulting signal appears less clear. Clean ECG signals can be obtained after processing the signals digitally, such as disclosed herein.

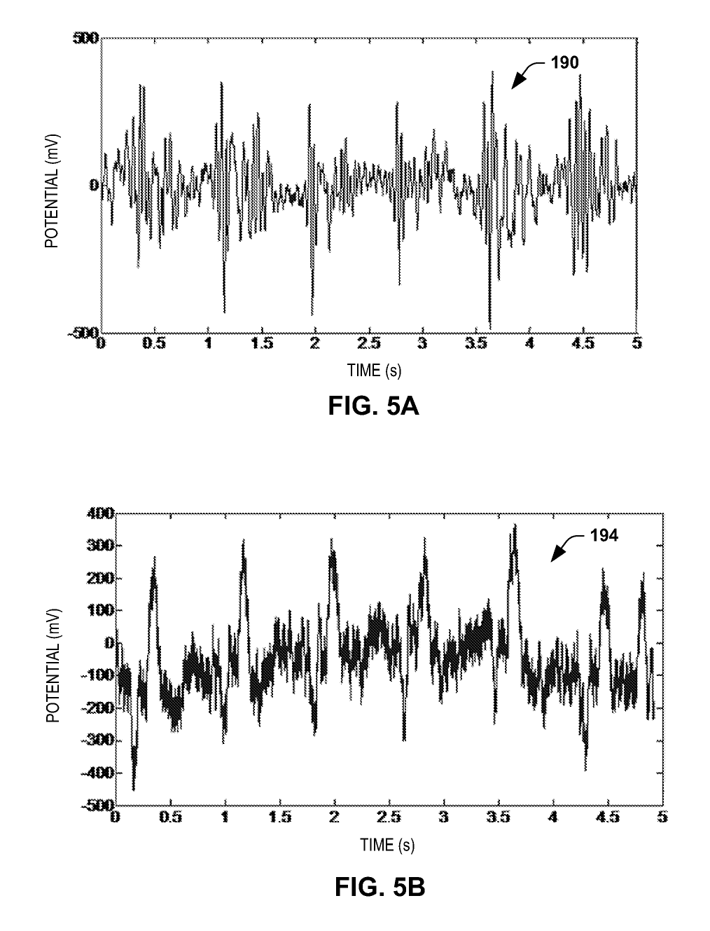

As disclosed herein, a non-contact electrode can be employed to acquire information of different parts of a subject's body. FIGS. 5A and 5B demonstrate waveforms 190 and 194 in which an electrode was positioned to detect ECG potential in the right chest and back of a subject. The signals shown in FIGS. 5 A and 5B were processed by an analog circuitry (e.g., the circuit 50 of FIG. 2) and digital filtering (e.g., the filter 42 of FIG. 1). For the example waveforms 190 and 194, the subject was seated in the chair as mentioned with respect to FIG. 3 and the distance between the subject's body and the sensor was fixed at 10 cm. Similar data acquired from left chest is shown in FIG. 4A.

In view of the foregoing, FIG. 6 depicts an example of a system 200 that can be implemented for providing an output that is indicative of fatigue of a subject, such as a driver (e.g., a driver of a motor vehicle such as a truck, bus or car). The system 200 can be implemented as computer readable instructions stored in a non-transitory computer readable medium (e.g., a hard disc drive, a flash drive, RAM, ROM or the like). The system 200 can receive inputs from one or more non-contact physiological sensors (e.g., electrode 12 or 52), such as disclosed herein.

In the example of FIG. 6, the system 100 receives a plurality of inputs, such as corresponding filtered, amplified signals (e.g., provided by respective analog circuits 14 of FIG. 1 or 50 of FIG. 2) which have been digitized. In the example of FIG. 6, the digital input signals are demonstrated as being from a non-contact EEG sensor, a non-contact ECG sensor and a non-contact EMG sensor. Other non-contact sensing can also be implemented, which may be more or less and different types of sensing than shown in FIG. 6. Each of the input signals can be processed by analog circuitry such as disclosed with respect to FIGS. 1 and 2. The analog signals can be converted to corresponding digital signals through appropriate analog to digital conversion and stored in memory (e.g., the memory 34 of FIG. 1). Thus the input signals from each of the respective sensors can correspond to a sampling of signals over a period of time.

As disclosed herein, the system 200 can be utilized to determine the physiological conditions of the subject in substantially real time. As used herein, the term substantially means that the function is intended to be designed to perform according to the term being modified performed; however variations due to signal processing speed, latency, variations in circuitry and the like can cause some variations (e.g., +/−10%).

Each of the input signals can be provided to a corresponding digital filter 202, 210 or 230 that may be programmed specifically for processing each physiological signal. For example, the signals corresponding to the EEG sensor can be provided to a digital filter 202 that is programmed with a pass band corresponding to EEG signals of interest, such as in a frequency band of 10 Hz to about 2 KHz. The digitally processed signal can be stored in a buffer or other memory and provided to an EEG calculator 204. The EEG calculator can be programmed to compute and indication of EEG wave forms. For example, the EEG calculator can be programmed to compute an alpha wave and a beta wave, indicated at 206 based upon the digitized signal provided by the digital filter. Other components of brain activity and of an EEG signal may also be derived by the EEG calculator. The EEG calculator 204 can provide the derived alpha and beta waveforms to a corresponding drowsiness correlation unit 208. In other applications, the computed EEG waveform can be provided to other devices, such as can vary depending upon how the waveforms are to be utilized. For example, the EEG calculator 204 can provide the derived waveforms to an output circuit that may present the EEG waveforms on a corresponding display or sent to a remote site for monitoring the respective subject.

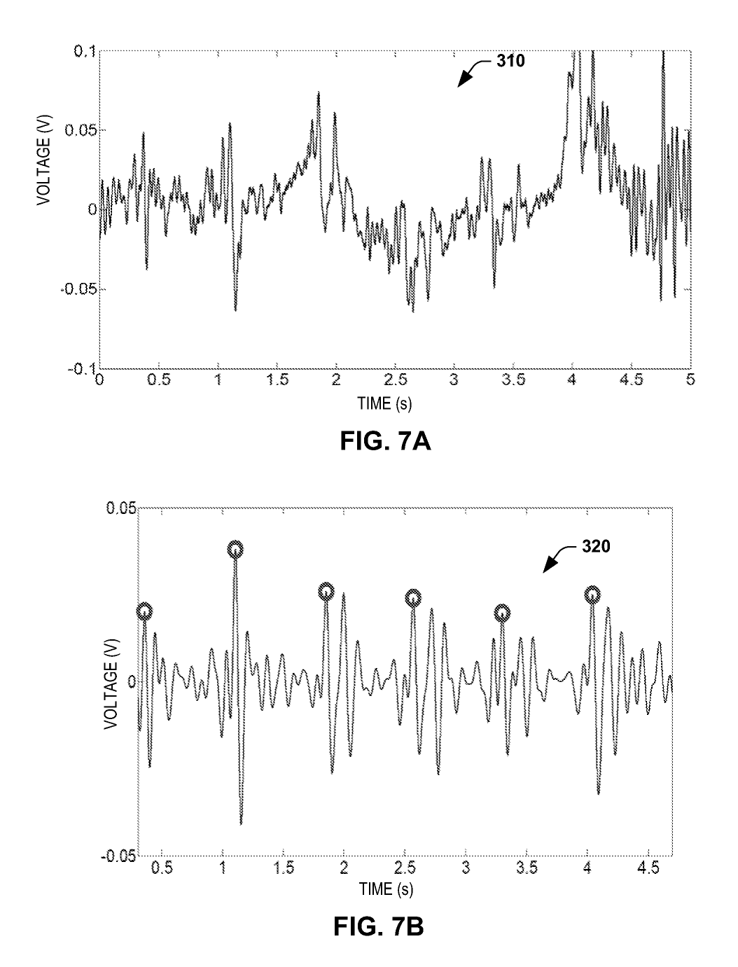

The digitized input signal from the ECG sensor can be provided to a corresponding digital filter 210. The digitized ECG signal can be provided to a variety of functions. In the example of FIG. 6, the digitized filtered ECG signal can be provided to an ECG waveform calculator 212. The ECG waveform calculator 212 can derive an ECG waveform based on the filtering and amplification performed on the input signal. An example ECG waveform 310 is as shown in FIG. 7A (see also, e.g., FIGS. 3A, 3B and 3C). The waveform provided by the calculator 212 can be constructed from a single non-contact sensor. Alternatively or additionally, the ECG waveform calculator 212 can compute an ECG waveform based on signals combined from multiple non-contact ECG sensors. The output of the ECG waveform calculator 212 can be provided to the drowsiness correlation unit 208 as well as to other outputs for other purposes.

The digital filter 210 can also provide the filtered signal to a heart rate calculator 214. The heart rate calculator 214 can compute the heart rate over a sampling time interval over a corresponding interval. The heart rate calculator 214 can include a peak detector to detect peaks in the signal content such as corresponding to the peak of the QRS complex of the ECG, such as peaks in the waveform 320 demonstrated in the example of FIG. 7B. The heart rate calculator 214 can also compute the heart rate based on time between adjacent peaks, which may be a time averaged indication from multiple heart beats. An instantaneous heart rate can also be provided by the heart rate calculator 214 to the drowsiness correlation unit. In other examples the, heart rate calculator 214 can compute the amplitude of T-wave, which is an interval of an ECG period.

The system 200 can also include a heart rate variation (HRV) calculator 220 that is programmed to compute heart rate variability for the subject. The heart rate variability can be computed based on the output from the heart rate calculator 214 such as based on the change in heart rate over a period of heart beats. The number of heart beats utilized in computing the heart rate variability can be set as a user programmable parameter. The HRV calculator 220 can compute the mean of the heart rate calculator over a plurality of heart rate values. Additionally, the HRV calculator 220 can compute the corresponding standard deviation of the heart rate variability. The corresponding mean and standard deviation values of the recent set of time average samples can be utilized to estimate the spectrum and distribution of the HRV, which can be provided to the drowsiness correlation unit 208. The heart rate variability calculator 220 can in turn provide the indication of heart rate variability to the drowsiness correlation unit 208.

As an example, the frequency of normal ECG usually ranges in 0.01-100 Hz, while the energy concentrates in about 5-45 Hz. After signal acquisition and A/D conversion, several sources of noise can be added into the original ECG signal, including EMG interference, frequency interference and baseline drifts. EMG signal can be caused by human behavior and muscle contraction, and can It ranges in 5˜2 kHz. Frequency interference results from power system, the frequency of which is 60 Hz. Baseline drift is led by low frequency interference, like the movement of electrode and breathing. The frequency can be about 0.05˜2 Hz.

Since the magnitude of interference due to such noise can be as high as, or even higher than the detected ECG signal, robust algorithm can be employed to de-noise. As one example, the signal processing (e.g., signal processing 38 of FIG. 2) can utilize a non-linear wavelet transform thresholding to decimate noise. Other techniques for noise reduction can also be utilized. Noise reduction can be implemented in hardware, software (e.g., instructions stored in memory and executed by a processor) or as a combination of hardware and software.

Non-linear wavelet transform thresholding is also called wavelet shrinkage. Wavelet shrinkage is to decompose the time-domain signal into different frequency levels, choose different thresholds in these levels, and then reconstruct the residuals in order to get the rational original signal. The energy of original signal concentrates in the low-frequency part which can reflect the shape, while the high-frequency part contains the details as well as noise. In different signal levels a certain threshold can be selected to decimate noise in each level. The different levels can be combined together to obtain the original signal.

As an example, the steps for implementing wavelet shrinkage (e.g., implemented by signal processing method 38 of FIG. 2) can be as follows:
(a) Calculate the wavelet transform of signal mixed with noise. According to Mallet decomposing algorithm, choose appropriate wavelet and level, decompose the whole signal and determine the wavelet coefficient for each level.
(b) Process the threshold of wavelet coefficient. The threshold processing can include hard threshold or soft threshold. In one example, soft threshold method can be used.
(c) Reconstruct the signal components in different frequency ranges. The signal obtained after this step will be the signal after noise decimation.

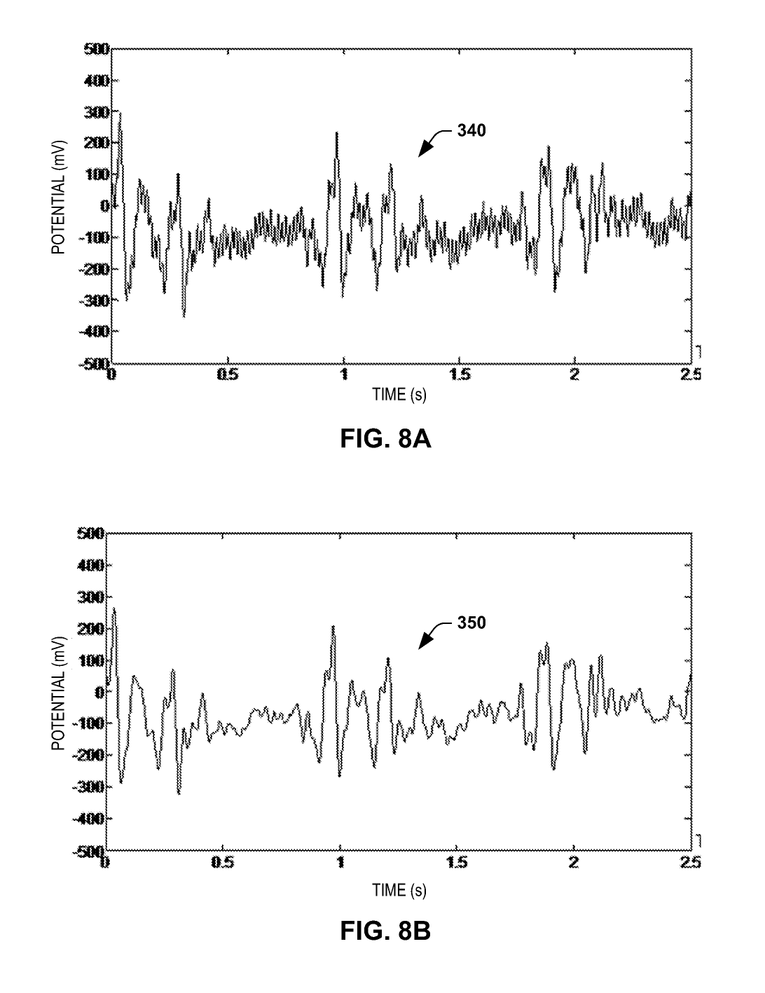

The effectiveness of the wavelet shrinkage can depend on selecting the appropriate wavelet base and value of threshold. As an example, the signal processing can utilize wavelet base symlet 8 and level 8. The signal shown in FIG. 6B is processed and illustrated through the algorithm as described above. The optimized threshold should be right above the magnitude of noise, so heuristic method can be used. A threshold of d6, d7 can be controlled (e.g., manually or automatically) in order to contain the frequency interference. As one example, the thresholds of d6, d7 are 26.214 and 443.794, respectively. After choosing rational thresholds, the various components are composed together, and the physiological signal can be recovered. The relative high frequency interference has been decimated after processing. FIGS. 8A and 8B demonstrate examples of the physiological signal before and after signal processing, respectively.

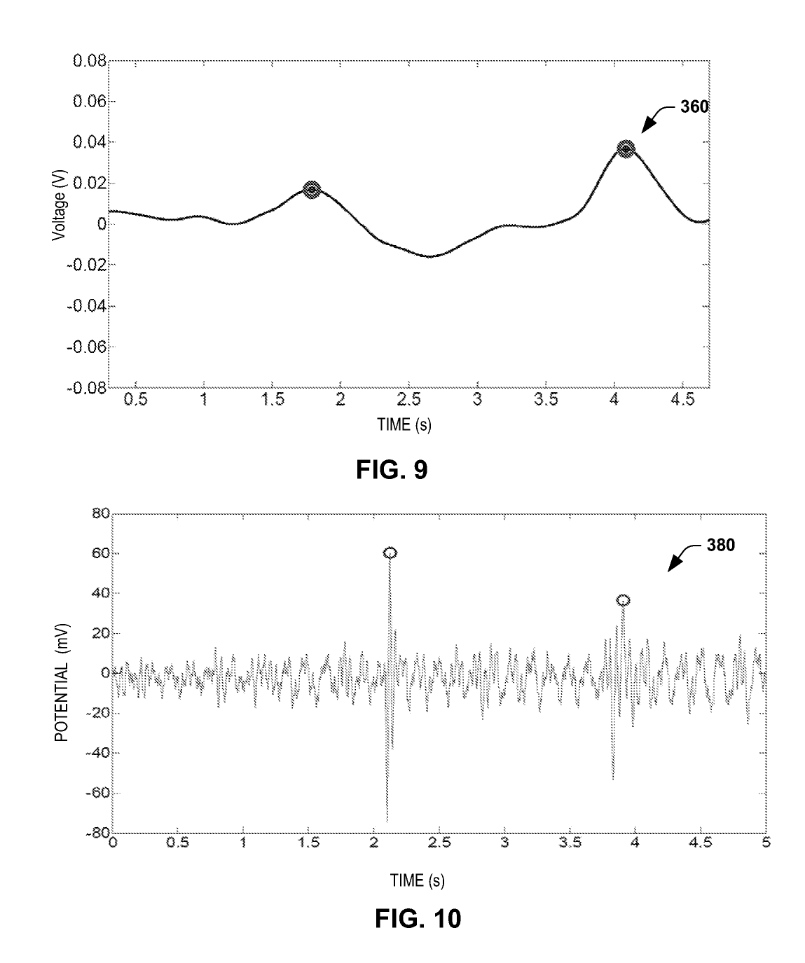

A breathing rate calculator 222 can also compute breathing rate based on the digitized ECG sensor signal. For example, the breathing rate calculator 222 can include a baseline extraction component 224 programmed to analyze the input signal from the ECG sensor and extract the corresponding baseline component thereof. The breathing rate calculator also includes a high pass filter 226 to pass the high frequency components of the signal and remove the lower frequency components. FIG. 9 demonstrates an example of a breathing signal 360 showing peaks between breaths from which the breathing rate can be calculated. The signal 360 can be derived (e.g., by baseline extraction and HP filtering) from the example ECG signal shown in FIGS. 7A and 7B. When the subject breaths during signal acquisition, the baseline of ECG signal will fluctuate (as shown in FIG. 7A). The pulses in the ECG signal depicted in FIG. 7B are indicative of breathing frequency. For instance, the number of pulses in a fixed interval can be counted to detect breathing frequency, which can be used further to compute its derivative for a change in breathing rate over time. The breathing rate calculator 222 in turn provides an output indicative of the breathing rate to the drowsiness correlation unit 208.

The system 200 also includes a digital filter 230 that is programmed to process the digitized EMG sensor signals and provide the corresponding filtered indication of the EMG signal. For example, the digital filter 230 can be programmed to provide a pass band in a range from about 10 Hz to about 10 KHz generally corresponding to the frequency range of a typical EMG signal. The filter of the digitized signal can be provided to a movement calculator 232 that can be utilized to compute an indication of the muscle activity that is encoded in the input signal from the EMG sensor. As one example, the EMG calculator 232 can compute an indication of eye blinking (or other) activity for a subject. The indication of the eye blinking can be provided as a value or it can be provided as a series of values that can correspond to an eye blinking or muscle activity waveform. The movement calculator 232 can provide the indication of muscle activity as another input to the drowsiness correlation unit 208. While the examples of FIG. 6 demonstrate a monitoring system that correlates EEG, ECG and EMG signals detected via non-contact sensors, the system can receive other types of signals such as disclosed herein or otherwise.

FIG. 10 is a graph demonstrating an example waveform 380 corresponding to a muscle activity signal that has been processed (e.g., by the movement calculator 232 and filter 230) based on EMG signals associated with eye blinking movement. Signal peaks for each time the eye blinks can be identified via peak detection, such as disclosed with respect to the ECG signal processing. The EMG activity due to eye blinking can correspond to distinctive pulse responses in the bioelectric signals detected by the non-contact sensor that is positioned near the subject's eyes. The inputs provided to the correlation unit 208 can characterize possible fatigue or fatigue onset for the subject.

The drowsiness correlation unit 208 can be programmed to set one or more thresholds 240 to determine an indication of drowsiness for the subject. The one or more thresholds 240 can be applied to each of the input signals individually to determine if the signal itself indicates a potential likelihood for drowsiness. The thresholds can be established based on empirical studies, research or other data collected related to determining drowsiness and for predicting onset of drowsiness, for example.

The drowsiness correlation unit 208 can also apply weighting 242 to the different inputs depending upon the expected reliability of the inputs provided to the drowsiness correlation unit. The drowsiness correlation unit 208, for example, can perform fusion with respect to a combination of two or more of the individual inputs, corresponding to the detected EEG electrical signals, ECG electrical signals and EMG electrical signals, to provide an aggregate result based on which an indication of drowsiness can be provided. The indication of drowsiness can be stored in memory and a corresponding output (e.g., countermeasures or signaling) can be provided such as disclosed herein.

Additionally, for the application of monitoring driver fatigue, the drowsiness correlation unit 208 can combine the sensed parameters of a subject with other signals, such as parameters indicative of vehicle performance and operator activity. Examples of such other parameters can include speed, lane changing, maintenance of distance between the vehicle and road lines, rotational angle of the steering wheel or other parameters that can be sensed directly or indirectly from the vehicle operation. That is, the non-contact monitored signals can be fused with such other signals (e.g., corresponding to vehicle performance or driver behavior), from which efficient drowsiness detection and countermeasure control can be implemented.

In view of the foregoing, it will be appreciated that a non-contact sensor and sensor system have been described. The sensor system can provide a non-contact monitor means for detecting electrophysiological signals for a variety of applications as disclosed herein. In one example, electrical activity on a subject's body can be capacitively coupled to a group of distributed non-contact sensors. The data acquired from such non-contact sensors can be used to monitor physiological condition of a driver, which can be utilized to determine an indication of driver fatigue.

As disclosed herein, the sensor can be designed to have high input impedance and by implementing filtering and amplification, including analog and digital means, output signals can be provided with good SNR characteristics at non-contact distances between sensor electrode and subject at 20 cm, and even up to 30 cm or greater. The signals can be obtained from a subject in real time and signal processing can be employed to remove the frequency interference, such as through wavelet shrinkage. The physiological information, such as the heart rate, heart rate variability, eye blinking, and breathing frequency, can be determined from the processed signals. The physiological information from the detected electrical activity can be aggregated and evaluated to characterize driver fatigue. The indication of driver fatigue can be utilized to trigger an output signal (e.g., for countermeasures) if the fatigue exceeds a predetermined threshold. The output signal can be stored memory (e.g., in a black box of the vehicle) and/or be utilized to provide a warning to the driver (e.g., audible, visual and/or tactile).

As will be appreciated by those skilled in the art, portions of the invention may be embodied as a method, data processing system, or computer program product. Accordingly, these portions of the present invention may take the form of an entirely hardware embodiment, an entirely software embodiment, or an embodiment combining software and hardware. Furthermore, portions of the invention may be a computer program product on a computer-usable storage medium having computer readable program code on the medium. Any suitable computer-readable medium may be utilized including, but not limited to, static and dynamic storage devices, hard disks, optical storage devices, and magnetic storage devices.

Certain embodiments of the invention are described herein with reference to methods, systems, and computer program products. It will be understood that functional and blocks and processing of signals can be implemented by computer-executable instructions. These computer-executable instructions may be provided to one or more processor of a general purpose computer, special purpose computer, or other programmable data processing apparatus (or a combination of devices and circuits) to produce a machine, such that the instructions, which execute via the processor, implement the functions described herein.

These computer-executable instructions may also be stored in computer-readable memory that can direct a computer or other programmable data processing apparatus to function in a particular manner, such that the instructions stored in the computer-readable memory result in an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified.

What have been described above are examples. It is, of course, not possible to describe every conceivable combination of components or methodologies, but one of ordinary skill in the art will recognize that many further combinations and permutations are possible. Accordingly, the invention is intended to embrace all such alterations, modifications, and variations that fall within the scope of this application, including the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

What is claimed is:

1. A non-contact physiological monitoring system, comprising:
a non-contact electrode configured to provide an input sensor signal based on electrical activity at a subject's body, the electrical activity being capacitively coupled to induce current on the non-contact electrode without contacting the surface of the subject's body, the input sensor signal corresponding to the induced current;
an instrument amplifier to amplify the input sensor signal to provide an amplified input signal;
a DC bias circuit configured as a high-pass filter to substantially remove DC offset in the amplified input signal and provide an offset-corrected signal;
a high order analog low pass filter in series with the DC bias circuit, the analog low pass filter being configured to pass frequency content below a predetermined cut-off frequency and to apply a gain factor to the offset-corrected signal and provide a corresponding analog output signal at an output representing the electrical activity at the subject's body, the gain factor being greater than about 500.

2. The system of claim 1, further comprising a shielded housing, the instrument amplifier, the DC bias circuit and the low pass filter residing within the shielded housing, the electrode residing outside of the shielded housing.

3. The system of claim 2, wherein the non-contact electrode is one of fixedly mounted to an exterior surface of the shielded housing or is connected to an input of the instrument amplifier via a shielded cable.

4. The system of claim 1, wherein the analog output signal comprises a voltage signal having a peak-to-peak amplitude that is greater than or equal to about 0.5 V.

5. The system of claim 4, wherein the instrument amplifier, the DC bias circuit and the low pass filter define an analog circuit, the analog circuit having an aggregate gain that exceeds 1000 such that the peak-to-peak amplitude of the voltage signal is greater than or equal to about 0.9 V for distances of up to about 25 cm between the non-contact electrode and the subject's body without saturation of the analog output signal.

6. The system of claim 1, wherein the low pass filter comprises at least two low-pass filter stages connected in series between the DC bias circuit and the output, each of the at least two low-pass filter stages having a filter order that is greater than one.

7. The system of claim 1, further comprising:
an analog-to-digital converter configured to convert the analog output signal to a corresponding digital signal; and
a processing device configured to perform signal processing on the corresponding digital signal to provide a digital output indicative of a physiological condition for the subject.

8. The system of claim 7, wherein the processing device further comprises a digital filter programmed to pass frequency content within at least one pass band

9. The system of claim 7, wherein the physiological condition comprises cardiac activity for the subject, the processing device configured to remove noise from the corresponding digital signal and provide the digital output as a processed signal representing an ECG waveform for the subject.

10. The system of claim 7, wherein the processing device further comprises a calculator configured to calculate a value indicative of at least one physiological condition from the corresponding digital signal.

11. The system of claim 10,
wherein the non-contact electrode comprises a plurality of electrodes, and
wherein the at least one physiological condition comprises heart rate, heart rate variations and breathing rate determined based on the electrical activity measured from the plurality of electrodes.

12. The system of claim 11, wherein the calculator is further programmed to derive an indication of fatigue of the subject based on the at least one physiological condition.

13. The system of claim 1, wherein the non-contact electrode, the instrument amplifier, the DC bias circuit and the low pass filter are mounted within a vehicle.

14. A system comprising:
a plurality of non-contact electrodes, each of the electrodes being configured to capacitively couple with an adjacent region of a subject's body that is spaced apart from the respective electrode and to provide a respective output signal corresponding to electrical activity sensed at the adjacent region via the capacitive coupling;
an analog circuit configured to amplify and filter each respective output signal and provide a respective analog output signal for each of the non-contact electrodes;
a processing device configured to process each analog output signal, the processing device comprising:
a digital filter programmed to filter a digital representation of each analog output signal and provide processed signals corresponding to each of the analog output signals;
a calculator to determine a plurality of physiological conditions for the subject based on the processed signals; and
an output generator configured to provide an output based on the plurality of physiological conditions for the subject.

15. The system of claim 14, wherein the plurality of physiological conditions of the subject comprise at least two of heart rate, heart rate variations, breathing rate, eye blinking and brain activity.

16. The system of claim 14, wherein the analog circuit for each of the plurality of electrodes further comprises:
an amplifier to amplify the output signal from a respective one of the plurality of electrodes to provide an amplified input signal;
a DC bias circuit configured as a high-pass filter to substantially remove DC offset in the amplified input signal and provide an offset-corrected signal;
a high order low pass filter in series with the DC bias circuit, the analog low pass filter being configured to pass frequency content below a predetermined cut-off frequency and to apply a gain factor to the offset-corrected signal and provide a corresponding analog output signal representing the electrical activity at the subject's body, the gain factor being greater than about 500.

17. The system of claim 16, wherein the amplifier, the DC bias circuit and the low pass filter collectively define the analog circuit, the analog circuit having an aggregate gain that exceeds 1000 such that a peak-to-peak amplitude of the voltage signal is greater than or equal to about 0.9 V and without saturation for distances of up to about 25 cm between the electrode and the subject's body.

18. A non-contact method for monitoring physiological conditions, comprising:
inducing electrical current on at least one electrode, which that is spaced apart from an adjacent region of a subject's body, via capacitive coupling between the respective electrode and the adjacent region of the subject's body;
receiving at least one electrical signal at an input corresponding to the induced electrical current;
amplifying and filtering the at least one electrical signal in the analog domain and providing a corresponding analog output signal in which DC bias has been substantially removed as to mitigate saturation of the corresponding analog output signal for a distance between the at least one electrode and the adjacent region of the subject's body that is up to about 30 cm;
digitally filtering a digital representation of the corresponding analog output signal to remove noise and providing a processed signal corresponding to the corresponding analog output signal;
determining at least one physiological condition for the subject based on the processed signal; and
generating an output based on the at least one physiological condition determined for the subject.

19. The method of claim 18, wherein the at least one electrode comprises a plurality of electrodes distributed in a non-contact relationship with the subject's body to monitor electrical activity,
wherein the amplifying and filtering, the digitally filtering and the determining are performed for signals from each of the plurality of electrodes based on which a plurality of physiological conditions are determined for the subject, and
plurality of physiological conditions.

20. The method of claim 18, wherein the amplifying and filtering in the analog domain are performed to provide an aggregate gain that exceeds 1000 and the corresponding analog output signal has a peak-to-peak amplitude that is greater than or equal to about 0.5 V for distances of up to about 25 cm between the electrode and the subject's body.
